(19) United States
(12) Patent Application Publication
(10) Pub. No.: US 2023/0399752 A1
(43) Pub. Date: Dec. 14, 2023

(54) Commercially Viable Biosensor Manufacture

(71) Applicant: Richard Postrel, Miami Beach, FL (US)

(72) Inventor: Richard Postrel, Miami Beach, FL (US)

(21) Appl. No.: 18/144,846

(22) Filed: May 8, 2023

Related U.S. Application Data
(60) Provisional application No. 63/339,493, filed on May 8, 2022.

(51) Int. Cl. C23C 28/00; C23C 14/14; C23C 14/58; C23C 16/40; C23C 16/455; C23C 16/56

(52) U.S. Cl. CPC C23C 28/322; C23C 14/14; C23C 14/5853; C23C 28/3455; C23C 16/45527; C23C 16/56; C23C 16/405

(57) ABSTRACT

This invention provides systems and methods for improved biosensor production resulting in enhanced surface adhesion with stronger dielectrics and reduced inter-sensor variance. The present invention greatly improves both the reliability and depth of data obtained. These greatly improved biosensors are ideally suited for the detection of early stage diseases and conditions such as cancers, pathogens, and toxic exposures. Practicing this invention allows for commercially viable manufacturing and the refurbishing of spent biosensors.

This invention provides systems and methods for improved biosensor production resulting in enhanced surface adhesion with stronger dielectrics and reduced inter-sensor variance. The present invention greatly improves both the reliability and depth of data obtained. These greatly improved biosensors are ideally suited for the detection of early stage diseases and conditions such as cancers, pathogens, and toxic exposures. Practicing this invention allows for commercially viable manufacturing and the refurbishing of spent biosensors.

These graphene/single wall carbon nanotube coated biosensors comprise an electronically responsive chip with sensing portions providing a signal (electronic variation) caused by molecules in close proximity, but not bound to highly sensitive, highly selective biosensor surfaces. The chips require ability to transmit the signal from the sensing surface to the electronic processing components for digitization and machine processing. The chips must respond to the electronic variations of the sensor surfaces and relay the changed signal for computational analysis. Between the biosensing surfaces and the electronic processing the electronic base chip of the present invention conveys the electronic signal from the compounds in close proximity to the sensing surface to the signal processing systems. Many chips are known to respond to electronic changes, however, the chips of the present invention are specially designed for use underneath a carbon based, preferably single wall carbon nanotube (SWNT) coated chips. The chips of the present invention provides a more uniform surface with stronger more consistent dielectrics for reliable highly responsive biosensors.

These highly sensitive chips display distinguishing behaviors when exposed to volatile organic hydrocarbons (VOCs). Consistent surface bonding and presentation of functionalized bio-sensor material to the gaseous emissions being analyzed is required. These biosensor chips require a supportive base upon which electronic sensors are situated. A silicon base is a preferred embodiment that is commonly used in the art. Silicon or similar supportive structures can be processed by thermal oxidation, e.g., heating to about 900° C. followed by exposure to oxygen. A thickness ranging from about 100 nm to 400 nm is considered adequate. A SiO₂ layer of approximately 280 nm is convenient and common in the art. A range from about 2 nm to 10 nm, for example, a 5 nm Cr layer is added followed by ˜40 nm (acceptable range: ˜15-70 nm, more preferably, ˜20-60nm, and still more preferably, ˜30-50 nm) palladium or other metal of the platinum group coating as a gate metal electrode. When changing layer compositions or introducing precursors, cleaning with one or more solvents and plasma etching is used to remove impurities. An organic solvent followed by O₂ plasma stripping to remove residuals and contaminating hydrocarbons introduced into the chamber at about 40 to 60° C., or about 50° C. for about 5 to 15 min decontaminates the chip surface between stages.

The sensor chips of the present invention feature an electronic signal produced when an activated/functionalized surface is altered by one or more compounds flowing over or pausing proximal to the functionalized surface. The invention described herein embodies three chip formation protocols that each have their own advantages. Each of the three produces a superior surface for interacting with graphene compositions such as single walled carbon nanotubes (SWNTs) and their functionalizing decorations. The result is a superior sensing chip available for assessing and characterizing patterns of volatile organic hydrocarbons (VOCs) that are emitted by living organisms suffering from or managing disease. Specific VOC emissions correspond to specific diseases and/or conditions. Accurate pattern recognition of these emissions can be used to identify a disease or disease status.

A chip presenting with the desired properties of durability, consistent compound recognition, high sensitivity and high selectivity can be formed on a silicon or similar base. Such formations are well-known in micro-electronics. In a first preferred embodiment, a physical vaporization process is used to form a thin ˜3-10 nm layer of metallic aluminum onto a silicon chip base. Such thin aluminum interfacing agent can formed by vaporizing metallic Al from a slug, strip, pellet, plug, or the like from a receptacle inside a vacuum chamber (˜10⁻⁶ torr). The slug is vaporized into the chamber for example by resistive heating or electron beam excitation. The resultant vapor sprays onto chip wafer to coat it with aluminum. During process development in a given apparatus, a quartz crystal monitor may be used to measure thickness to determine protocols for depositing the selected thickness. In routine practice the thickness is then controlled by timing the opening of a shutter barricade that controls the slug receptacle access to the chamber. The Al (˜3 nm) is allowed to interact with air (˜20% O₂) or other oxygen source to convert the Al to an aluminum oxide (Al₂O₃) nanocrystalline film surface acceptable to hafnium compound deposition.

Atop the Al₂O₃, a hafnium precursor compound (precursor A) is introduced in alternating atomic layer deposition cycles (ALD) into the chamber with an oxygen donor compound (precursor B). Precursor B strips the non-hafnium atoms from the precursor A and replaces them with oxygen. The atomic layering cycles continue alternating hafnium precursor A and the stripping precursor B to build up multiple even layers of dielectric, HfO2 in this example, to a desired thickness (often a factor of the expected voltage). The chip is annealed to regularize the surface before overcoating with a graphene, preferably a coiled graphene such as a SWNT. The conductive carbon coating is then treated with a selective biomolecule (functionalizing compound) such as a DNA to differentiate the receptor responses beyween different patterns of VOCs. Different DNAs or other functionalizers will present different responses with different electron clouds.

The DNA or other functionalizing agent electron clouds interact with and loosely bond/adhere to the carbon layer. The functionalizing compounds remain weakly bonded until a chip might be treated in scheduled maintenance or if damaged. These chips can be returned to a shop, laboratory or foundry to refurbish, remove, and/or replace the functionalyzing surface or if desired the SWNT layer and the functionalyzing decoration(s).

When a molecule such as one or more VOC molecules comes in close proximity to the functionalizing molecules on a chip, the electrons surrounding the surface of the VOC repel electrons on the functionalizing surface molecules. Less electron dense portions of a VOC will draw electrons from the decoration molecule farther from the carbon/SWNT surface. The electronic changes on the surface molecules are detected and transmitted through the chip for analysis and characterization of the VOC pattern that can be specifically correlated to one or more diseases or conditions.

A second chip fabrication system of the present invention comprises using atomic layer deposition to form an Al₂O₃ interface or underlayment between an eventual hafnium oxide capping surface and a silicon (or other) supportive substrate. The Al₂O₃ layer in this embodiment, formed using ALD, may be similar in thickness (e.g., 3 nm) to the vaporized aluminum deposition procedure or made thicker (e.g., ≥˜30 nm). Owing to the serial cyclic ALD depositing regimen, preparation time increases with increased thickness. These ALD layers will take on a more regular structure than the physical deposition formed Al/Al₂O₃ layer in the embodiment above. The reliably heat conductive aluminum oxide intermediate dielectric underlying a HfO2 sensor compound binding layer provides a bridge between the e.g., SiO₂ and HfO₂ with excellent thermal and electronic stability. For this and other embodiments of the invention, many possible support substrate surfaces are available. SiO₂, sapphire, PET, e.g., oxide or H-terminated substrate surface, and the like can act as a supportive starter substrate surface for many electronic chip, including ALD treated chip, applications. Al₂O₃ is a favored wetting agent or bridge for such starters. The current invention favors a metallic oxide substrate layer that can itself be deposited as a thin film on a more massive structural support. An Aluminum oxide(Al₂O₃) is a preferred initiating layer on such supports.

A third embodiment also features Al₂O₃ and HfO₂ with a more complex ALD protocol—wherein alternating temperatures are used for the layering.

A brief summary of the Atomic Layer Deposition (ALD) process is provided for context.

In ALD, a primary (first) layer, is deposited from a gas precursor A onto a chemically active, e.g., oxide or H-terminated substrate surface. Many substrate surfaces are available. SiO₂, sapphire, PET, and the like can act as a supportive starter substrate surface for many ALD applications. The current invention favors a metallic oxide substrate layer that can itself be deposited as a thin film on the more massive support base. An Aluminum oxide(Al₂O₃) is a preferred initiating layer on the base.

Precursor A is allowed to react to completion, i.e., chemically and physically saturating the substrate surface. The substrate presents with a uniform coating of precursor A. Precursor A is flushed from the reaction chamber with a non-reactive (inert with respect to the precursor and chemically active surface) gas. Precursor B is introduced and allowed to react with the exposed active sites from the uniform deposition of precursor A. When all precursor A deposits are reacted with precursor B, precursor B is flushed with inert gas. Since the sites of each layer are saturated in each cycle, a uniform one molecule thick surface is presented for accepting the next precursor.

ALD is applicable to many compounds for different designed outcomes. For example, ALD is used to produce classes of compounds including, but not limited to: oxide dielectrics, oxide conductors or semiconductors, nitride dielectrics or semiconductors, metallic nitrides, fluorides, sulfides, etc. II-VI semiconductors, II-VI based phosphors, III-V semiconductors, etc. Oxide dielectrics in the art are numerous including, but not limited to: BiₓSi_{y}O, SrTa₂O₆, SrBi₂Ta₂O₉, YScO₃, LaAlO₃, NdAlO₃, Al₂O₃, TiO₂, ZrO₂, HfO₂, Ta₂O₅, Nb₂O₅, Sc₂O₃, LaYbO₃, Er₃Ga₅O₁₃, Y₂O₃, MgO, B₂O₃, SiO₂, LaScO₃, LaLuO₃, Er₂O₃, Tm₂O₃, Yb₂O₃, Lu2O₃, SrTiO₃, BaTiO₃, PbTiO₃, PbZrO₃, BiₓTi_{y}O, GdScO₃, Sm₂O₃, EuOₓ, Gd₂O₃, GeO₂, La2O₃, CeO₂, Dy₂O₃, HO₂O₃, PrOₓ, Nd₂O₃, etc. Oxide conductors or semiconductors include, but are not limited to: CuOₓ, FeOₓ, CrOₓ, CoOₓ, MnOₓ, ZnO:Ga, RuO₂, RhO₂, IrO₂, Ga₂O₃, VO₂, In₂O₃:Sn, In₂O₃:F, In₂O₃:Zr, SnO₂, In₂O₃, SnO₂:Sb, Sb₂O₃, ZnO, ZnO:Al, ZnO:B, V₂O₅, WO₃, W₂O₃, NiO, etc. Oxides of rhodium, iridium palladium osmium, ruthenium, and platinum are quite common in chip architecture. Other ternary oxides include, but are not limited to: LaCoO₃, LaNiO₃, LaMnO₃, La1-ₓCaₓMnO₃, etc. Nitride dielectrics or semiconductors include, but are not limited to: BN, AlN, GaN, InN, LaN, LuN, Zr₃N₄, Hf₃N₄, Si₃N₄, Ta₃N₅, Cu₃N, etc. Metallic nitrides include, but are not limited to: CoₓN, SnₓN, TiN, Ti—Si—N, Ti—Al—N, TaN, NbN, MON, WNₓ, WNₓC_{y}, etc. II-VI semiconductors include, but are not limited to: CaS, SrS, BaS, CdS, CdTe, MnTe, HgTe, ZnS, ZnSe, ZnTe, etc. II-VI based phosphors include, but are not limited to: ZnS:M, where M=Mn, Tb,Tm; CaS:M, where M=Eu, Ce, Tb, Pb; SrS:M, where M=Ce,Tb, Pb; etc. III-V semiconductors include, but are not limited to: GaAs, AlAs, AlP, InP, GaP, InAs, etc. Other semiconductors include, but are not limited to: PbS, SnS, In₂S₃, Sb₂S₃₂, CuₓS, CuGaS₂, WS₂, SiC, Ge₂Sb₂Te₅, etc.

The present invention discloses exemplary systems and methods for the construction of biosensor arrays with improved dielectrics, and adhesion characteristics relevant to coating with two dimensional carbon structures, including two dimensional carbon structures formed into SWNTs. This invention utilizes a multi-layer deposition process with an Al₂O₃ base supporting a hafnium oxide (HfO₂) dielectric to which the carbon adheres. The examples disclosed in the current application feature oxides of aluminum and hafnium. With appropriate tweaks in timing, flow rates temperatures, second precursors, etc., the principles of the present intervention are adaptable to mimic these ALD formed layers. Chambers specialized for size, temperature, volumes per time of gases fed, ease of cleaning—including self cleaning capacities, number of precursors and/or other gases, low purchase or operational costs are commercially available to the ALD practitioner. Different chambers may require compensatory adaptations to match details in the disclosed examples.

With special regard to the HfO₂ based top layers exemplified in further detail below, biosensor arrays that feature planar/single layer carbon structures such as SWNTs and graphene have been popularized as tools for sensing molecules in a gas phase. The sensing elements are generally coated with a selective dopant molecule such as a nucleic acid. The dopant interacts with proximal molecules to alter the electric field of the carbon layer. The dielectric superposed by the conductive carbon layer transmits the electric field alteration to the conductive substrate below, e.g., by altering current flow or potential difference. A predictable and consistent response to a given set of the nearby interacting compounds is important for sensor sensitivity and selectivity. These response patterns are analyzed and collated to correspond a specified+disease or condition

Current best practices for layering metallic oxides, such as the preferred HfO₂ , as thin films involve performing ALD. The objective biosensing elements of the present invention demonstrate improved signal stability and sensor durability in comparison to the prior art.

### EXAMPLES

In the primary embodiment a support chip, e.g., a silicon wafer chip is sprayed with Al vapor to form a coating ˜1-5 nm, preferably ˜3 nm which is oxidized to form an interface “wetting” layer between silicon and the hafnium-based dielectric. This and other preferred embodiments feature fabrication systems using , for example, the silicon chip surface substrate as described above. In this embodiment, the support substrate is chemical vaporization coated (CVD) with a thin, e.g., ˜1-10 nm, ˜2-7 nm, ˜5 nm, ˜3 nm, layer of metallic aluminum. The deposited Al is allowed to interact with air (˜20% O₂) to produce an aluminum oxide (Al₂O₃) surface acceptable to hafnium precursors. As the Al layer approaches and even may be made to exceed 10 nm, time for interaction with an oxygen source may be extended. While not especially preferred in these examples, other aluminum deposition protocols are available in the art to form nanocrystalline sometimes referred to as essentially amorphous Al₂O₃.

The Al chemical vapor deposition preferably produces, for example, a 3±1 nm aluminum base, a 5±1 nm aluminum base, a 10±1 nm aluminum base, etc. More stringent quality control may produce chips with an x±0.5 nm base coating. Chips with the ˜3±1 nm acceptance criteria are acceptable for successful later treatment to add the hafnium dielectric for topping with the nanoselective sensing coatings on the chip surface.

The metallic aluminum surface is processed by oxidizing in atmospheric gas, oxygen at reduced or ambient pressure, or oxygen or ozone fed into the chamber with a feeder gas for a few seconds (˜3-10 sec is adequate for a coating of ˜3-10 nm thickness) with minimal heat in the chamber, e.g., <250° C., <200° C., <150° C., <100° C., <80° C. The dielectric, preferably a hafnium dielectric, is then formed on the Al₂O₃ under atomic layer deposition conditions (see discussions above and below). An annealing step finishes the dielectric surface improve stable performance and interaction to accept planar carbon such as SWNTs. The SWNTs are then coated with a functionalizing compound, preferably a nucleic acid such as a DNA up to about 30 nucleotides in length.

The preferred dielectric coating comprises multiple atomic deposited layers of HfO₂. A hafnium precursor is introduced into the reaction chamber which is heated above ambient to about 100° C. up to 400° C. More preferably the chamber temperature is between about 150° C. to 300° C., or still more preferably, a temperature between about 200° C. to 250° C. is maintained. Precursor is fed into the chamber for several seconds before flushing with inert (non-reactive) gas before introducing the second precursor compound.

Atop the aluminum interface layer, in this example, the dielectric is HfO₂ formed using ALD. A hafnium precursor (precursor A), tetrakis(ethylmethylamino)hafnium in this example, is added to a nitrogen (inert) stream while the chamber temperature is maintained at about 250° C. After the Al₂O₃ sites are saturated with hafnium precursor, the nitrogen stream continues, devoid of the precursor, to flush unspent precursor and reaction residues. Precursor B is then introduced. E.g., ozone, H₂O₂, and/or H₂O is added to the nitrogen stream to convert the hafnium atoms on the surface to HfO₂. The cycling continues over the now HfO₂ with reintroduction of hafnium precursor A. After the HfO₂ sites are saturated with the hafnium precursor, the nitrogen stream continues devoid of the precursor to flush unspent precursor and reaction residue. Precursor B is introduced. Repeatedly precursor A is added, then precursor B to the nitrogen stream in cycles to convert the hafnium compositions from A on the surface to HfO₂. The empty nitrogen stream continues to flush precursor B and reaction residue from the chamber. The cycling of alternating precursors A and B continues to produce a HfO₂ surface of desired thickness. In this example, temperature in the chamber is maintained at about 250° C. throughout the cycling reactions. The cycling of precursor A and ozone is repeated about 100 to 1500 times, preferably about 600 to 1000 times, more preferably about about 600 times to produce a hafnium oxide base layer for receiving the sensing layer about 10 nm to 100 nm, preferably, about 20 nm to 70 nm, more preferably about 30 nm to 50 nm thick, or ˜40 nm thick. The surface is prepared for accepting the selective biosensing compounds in accordance with customary practice, e.g., by annealing, flushing, and washing.

A 10 nm Al₂O₃:10 nm HfO₂ chip coating is one preferred embodiment. Other embodiments including, but not limited to 10 nm Al₂O₃:10 nm HfO₂ chips, 10 nm Al₂O₃:20 nm HfO₂ chips, 3 nm Al₂O₃:10 nm HfO₂ chips 10 nm Al₂O₃:30 nm HfO₂ chips, 5 nm Al₂O₃:20 nm HfO₂ chips, 3 nm Al₂O₃:30 nm HfO₂ chips, 3 nm Al₂O₃:50 nm HfO₂ chips, 5 nm Al₂O₃:40 nm HfO₂ chips, 10 nm Al₂O₃:60 nm HfO₂ chips, 3 nm Al₂O₃:1600 nm HfO₂ chips, etc., may be chosen for manufacturing, packaging, durability, etc, considerations. The ˜3, 5, and 10 nm Al₂O₃ layers exemplified above or a thicker surface, e.g., 20, 25, 30 nm, etc., might be paired with HfO₂ thicknesses such as the ˜10, 20, 30, 40, 50, 60 nm thick HfO₂ layers in the above examples in a mix or match basis. Increased thicknesses, that may include thicknesses beyond those listed above, allow for higher voltage operation. Once prepared, SWNT adheres to the annealed HfO₂ surface prior to functionalyzing with a bio-molecule such as DNA. An array comprising several chips with different functionalyzing compounds is a preferred assembly using these functionally decorated chips.

Annealing is a conventional process for regularizing the surface, freeing it of oxygen gaps or irregularities. A surface anneal delivers an even, regular surface for bonding or interfacing with molecules at the surface. A robust deep anneal can improve dielectric behavior. The annealing process fills chemical gaps where incomplete oxidation may leave pores or irregular electronic behavior. Two annealing protocols are exemplified here as examples. One high temperature, one lower temperature. Several different annealing procedures can be found in the literature. At least one of these procedures is preferred to improve the density and reduce the variance of SWNT adherence to the HfO₂ surface.

In a higher temperature protocol, an oxygen source, including, but not limited to water vapor, O₂, O₃, etc., is introduced in the presence of sustained heating to between ˜500° C. and 1000° C. One common practice to anneal at about 900° C., for 60±15 min. This produces a HfO₂ finish with excellent adherence for the carbon bridge (two dimensional or rolled graphene) and with a more regular carbon surface better functionalization with selective biomolecules.

A lower temperature annealing protocol involves treating the chips in the presence of an oxygen source, preferably high purity oxygen, e.g., 99.995% O₂, at reduced pressure (<760 torr, e.g., ˜10⁻¹ torr or ˜10⁻⁴ atm) with sustained heating to between ˜100° C. and 200° C. for at least ˜15 to 60 min, preferably ˜45 to 60 min. This protocol also forms a HfO₂ finish with excellent adherence for the carbon bridge and subsequent functionalyzing compounds.

In a second preferred embodiment, an underlayment of Al₂O₃ again provides a substrate for the HfO₂ graphene dielectric and signal transmittal layers. In one embodiment a 30 nm Al₂O₃ interface base is formed using ALD. After the last aluminum cycle, cycles involving coating with the hafnium precursor to produce a HfO₂ dielectric between, e.g., 10 and 100 nm, preferably about 20 to 50 nm proceeds.

For forming an Al₂O₃ base under ALD, trialkylaluminum precursors, e.g., trimethyl-, triethyl-, etc., are available commercially often used as precursor materials. Other precursors include, but are not limited to aluminum acetylacetonate, multimers of aluminum isopropoxide, aluminum 2-ethylhexanoate, trialkyldialuminum trialkoxides (e.g., triethyldialuminum triisopropoxide and triethyldialuminum tri-sec-butoxide), tris(dimethylamido)aluminum(III), etc. The oxidized aluminum is then coated with a dielectric formation of HfO₂. Hafnium tetrahalides are available precursor compounds. Tetrakis(ethylmethylamino)hafnium is another commonly used precursor. The Hf precursor is alternated with an oxygen containing precursor to build the HfO₂ dielectric layer.

In ALD, the practitioner can select a preferred precursor A for each metallic oxide. Many precursors for aluminum have been used. These include, but are not limited to: Tris(2,2,6,6-tetramethyl-3,5-heptanedionato)aluminum (Al(C₁₁H₁₉O₂)₃), hexakis(dimethylamino)dialuminum (C₁₂H₃₆Al₂N₆), aluminum acetylacetonate (Al(CH₃COCHCOCH₃)₃), aluminum ethoxide (Al(OC₂H₅)₃), aluminum s-butoxide (Al(OC₄H₉)₃)\, aluminum chloride (AlCl₃), aluminum iodide (AlI₃), aluminum hexafluoroacetylacetonate, (Al(CF₃COCHCOCF₃)₃), tris(dimethylamido)aluminum(III) (Al(N(CH₃)₂)₃), dimethylaluminum i-propoxide (CH₃)₂Al(OC₃H₇)), trimethylaluminum ((CH₃)₃Al), etc. Similarly many precursor As for hafnium, including, but not limited to: tetrakis(diethylamido)hafnium(IV) (CH₂CH₃)₂N)₄Hf), hafnium(IV) chloride (HJfCl₄}, hafnium(IV) ethoxide (Hf(OC₂H₅)₄), tetrakis(dimethylamido)hafnium (CH₃)₂N)₄Hf), hafnium(IV) chloride (HfCl₄), tetrakis(ethylmethylamido)hafnium (C₂H₅)CH₃N)₄Hf), Hafnium(IV) t-butoxide (H(OC(CH₃)₃)₄), bis(pentamethylcyclopentadienyl)hafnium dichloride (((CH₃)₅C₅)₂HfCl₂), bis(ethylcyclopentadienyl)hafnium dichloride ((C₅H₄(C₂H₅))₂HfCl₂), bis(cyclopentadienyl)hafnium dichloride, (C₅H₅)₂HfCl₂), bis(cyclopentadienyl)dimethylhafnium ((C₅H₅)₂Hf(CH₃)₂), etc., can be selected by the practitioner.

Aluminum precursor A is added to a nitrogen stream while the chamber temperature is maintained at about 250° C. A clean nitrogen stream flushes precursor A from the chamber. Precursor B is then added to the stream before being flushed with nitrogen or other inert gas devoid of precursor. The ALD cycling is repeated about 200 to 300 cycles to form the Al₂O₃ base for hafnium addition. A base of ˜10 nm to 50 nm, more preferably ˜15 nm-40 nm, including ˜20 or 30 nm produces a solid support for the HfO₂ dielectric layer.

Atop this aluminum oxide interface layer, the HfO₂ layer is formed using ALD similar in procure used in the previous example.

In a third preferred embodiment, ALD is performed in two distinct formats. The first depositing format features an aluminum precursor for forming the Al₂O₃ underlayment. Then a HfO₂ layer is added to serve as a binding substrate for a conductive sensing layer such as graphene, preferably tubular graphene, more preferably as SWNTs. The practitioner can select a preferred precursor A for each metallic oxide. Many precursors for aluminum have been used. These include, but are not limited to: Tris(2,2,6,6-tetramethyl-3,5-heptanedionato)aluminum (Al(C₁₁H₁₉O₂)₃), hexakis(dimethylamino)dialuminum (C₁₂H₃₆Al₂N₆), aluminum acetylacetonate (Al(CH₃COCHCOCH₃)₃), aluminum ethoxide (Al(OC₂H₅)₃), aluminum s-butoxide (Al(OC₄H₉)₃)\, aluminum chloride (AlCl₃), aluminum iodide (AlI₃), aluminum hexafluoroacetylacetonate, (Al(CF₃COCHCOCF₃)₃), tris(dimethylamido)aluminum(III) (Al(N(CH₃)₂)₃), dimethylaluminum i-propoxide (CH₃)₂Al(OC₃H₇)), trimethylaluminum ((CH₃)₃Al), etc. Similarly many precursor As for hafnium, including, but not limited to: tetrakis(diethylamido)hafnium(IV) (CH₂CH₃)₂N)₄Hf), hafnium(IV) chloride (HJfCl₄}, hafnium(IV) ethoxide (Hf(OC₂H₅)₄), tetrakis(di-methylamido)hafnium (CH₃)₂N)₄Hf), hafnium(IV) chloride (HfCl₄), tetrakis(ethylmethylamido) -hafnium (C₂H₅)CH₃N)₄Hf), Hafnium(IV) t-butoxide (H(OC(CH₃)₃)₄), bis(pentamethylcyclopenta -dienyl)hafnium dichloride (((CH₃)₅C₅)₂HfCl₂), bis(ethylcyclopentadienyl)hafnium dichloride ((C₅H₄(C₂H₅))₂HfCl₂), bis(cyclopentadienyl)hafnium dichloride, (C₅H₅)₂HfCl₂), bis(cyclopenta-dienyl)dimethylhafnium ((C₅H₅)₂Hf(CH₃)₂), etc., can be selected by the practitioner.

Precursor A for the Al₂O₃ underlayment can be any aluminum precursor, but for this example, AlCl₃ is used. Tetrakis(ethylmethylamino)hafnium (C₁₂H₃₂HfN₄) is used as a hafnium example. HfCl₄ is easily substituted. To prepare the substrate surface to receive precursor A, a palladium conductive substrate surface is coated with an aluminum base layer using vapor deposition. Ozone is fed into the chamber to convert the aluminum to a reactive Al₂O₃ surface receptive to the precursor A, AlCl₃ in this example. The ozone is flushed from the system chamber using nitrogen. Precursor A is added to the nitrogen stream while the chamber temperature is maintained at about 250° C. After the Al₂O₃ sites are saturated, the nitrogen stream continues devoid of the precursor to flush unspent precursor and reaction residue. Precursor B is introduced. Ozone or water is added to the nitrogen stream to convert each top layer aluminum atom to Al₂O₃. An inert flush clears precursor B from the chamber. The cycling of AlCl₃ and ozone is repeated 50 to 400 times, preferably 100 to 300 times to produce an aluminum oxide base layer about 10 to 40, preferably, about 15 to 35, more preferably about 20 to 30 nm thick. A HfO₂ topcoat is then added.

Precursor A is flushed from the system with empty nitrogen and precursor B is again introduced. Precursor B and reaction residues are flushed from the system with the empty nitrogen stream. Chamber temperature is reduced to about 150° C. and the nitrogen feed gas is set to about 150° C. as it feeds precursor A into the chamber. After saturation, precursor A is flushed from the system chamber before introduction of precursor B. After saturation, precursor B is flushed from the system. Whereupon the chamber temperature and nitrogen feed stream temperatures are increased to about 200° C. or higher. The nitrogen stream at about 200° C. or higher feeds precursor A into the chamber. Post saturation precursor A is flushed from the system with empty nitrogen and precursor B is again introduced. Precursor B and reaction residues are flushed from the system with the empty nitrogen stream. Chamber temperature is reduced to about 150° C. The deposition cycles continue with alternate layers deposited at 150° C. and 200° C. Best results are obtained with a low temperature final cycle. At every deposit cycle, each precursor step is maintained at the assigned temperature for a time and precursor feed concentration to allow saturation of the available reactive sites. The smooth surface characteristic of low temperature deposition procedures results with fewer atomic impurities. The dielectric breakdown observed using higher temperature depositions is avoided with absence of the nanopores characteristic of such higher temperature deposition practices.

In chip manufacture freedom from undesired compounds or process residues is important. Accordingly after every photoresist post lithography step cleaning is desired. For example a rinsing with a solvent such as N-methyl-2-pyrrolidone (NMP) followed by a water rinse is included in the processing protocols. Additional or alternative wash steps may be used, such as acetone followed by 2-propanol. Tetrahydrofuran, dimethysulfoxide, dipropylene glycol dimethyl ether (DME), dimethylformamide (DMF), tetraoxaundecane, and N-butylpyrrolidone have been suggested as alternative solvents.

An oxygen cleaning (plasma etching) generally is employed for a cleaner surface. The specific practice is not an essential component of the invention. The oxygen plasma features energetic electrons with reactive oxygen atoms. These oxygen radicals oxidize the photoresist surface to react and volatilize the residuals as CO, CO₂, and H₂O. The contaminant compounds, chiefly hydrocarbons, are thus removed. Plasma, generally oxygen, but potentially enhanced with fluorine additives, is produced through exciting oxygen atoms with high energy electromagnetic treatment. The plasma then is allowed to react with (clean) the chip before the next stages in processing.

For illustrative purposes four practices are briefly highlighted. The first method involves generating or forming a plasma barrel ash wherein the wafer is surrounded by or immersed in plasma. In parallel plate plasma treatment, the plasma is generated above the wafer in an electric field and the electric potential draws the charged plasma particles towards and over the wafer. In some foundries, a remote plasma chamber is placed over the wafer(s) as a vacuum draws the plasma over and across the chips in a chip chamber. When a gentle treatment is desired, ozone can be formed by treating oxygen with uv light to surround and soak/bathe the chips. During development a spectrophotometer may be used to monitor HC emission wavelength(s) disappearance to determine the time the plasma treatment should continue in the chamber

Temperatures and times of treatment are not to be considered critical to forming the superior chips of the present invention. Time and temperature may be adapted to optimize fabrication in a specific chamber with different precursor compounds.

The chips of the present invention while stable, selective and sensitive can be refurbished. Preferably the chips incorporate a machine readable ePROM that identifies the chip construction including the functionalyzing compounds. With the ePROM, chips may be batch processed to re-functionalyze with the same or a different functionalyzing compound. Depending on state of performance, the compound may merely be refreshed or stripped and replaced with a different compound. The replacement compound may be selected for optimized detection for a different condition or disease. The replacement compound may result in improved performance, e.g., more selective, substrain distinguishing, faster performing, etc.

In more rigorous maintenance the carbon layer may be refreshed or replaced and then refunctionalyzed.

Where numeric values are indicated the expressions are not intended to be exacting. The values are expressed as approximations. For example unless precision is indicated, a reference to “5 to 7 days” is intended to indicate “about 5 to about 7 days” unless context would indicate otherwise. The presence of one approximation expression in a discussion is not intended to indicate values that are not expressly indicated as approximate are in fact precise.

1. A method for producing a highly sensitive, highly selective sensing chip, said method comprising:
depositing with physical layer deposition a layer of aluminum on a chip substrate, wherein thickness of said layer of aluminum is between 1 nm and 5 nm;
exposing said layer to an oxygen source wherein aluminum in said aluminum layer is oxidized to form Al₂O₃;
performing a plurality of atomic layer deposition (ALD) cycles to build a HfO₂ dielectric layer between about 20 nm and about 80 nm on the Al₂O₃;
annealing said chip;
applying a composition comprising single wall carbon nanotubes to form a coating on the HfO₂ dielectric; and
applying a sensitivity functionalizing compound to said coating.

2. A method for producing a highly sensitive, highly selective sensing chip, said method comprising:
depositing with physical layer deposition a layer of aluminum on a chip substrate, wherein thickness of said layer of aluminum is between 1 nm and 5 nm;
exposing said layer to an oxygen source wherein aluminum in said aluminum layer is oxidized to form Al₂O₃;
performing a plurality of atomic layer deposition (ALD) cycles to build a HfO₂ dielectric layer between about 20 nm and about 80 nm on the Al₂O₃;
annealing said chip;
applying a graphene composition to form a carbon composition coating on the HfO₂ dielectric; and
applying a sensitivity functionalizing compound to the carbon composition coating.

3. The method of claim 2 wherein said graphene comprises single wall carbon nanotubes (SWNTs).

4. The method of claim 1 wherein said layer of aluminum oxide is about 3±1 nm over its surface.

5. The method of claim 1 wherein said HfO₂ dielectric thickness is between about 10 nm and 70 nm.

6. The method of claim 1 wherein said HfO₂ dielectric thickness is between about 20 nm and 50 nm.

7. The method of claim 1 wherein said HfO₂ dielectric thickness is about 40 nm.

8. The method of claim 1 wherein said sensitivity functionalizing compound comprises nucleic acid.

9. The method of claim 8 wherein said nucleic acid comprises DNA.

10. The method of claim 1 wherein said atomic layer deposition (ALD) cycles are performed in a chamber with a temperature in a range about 200±50° C.

11. The method of claim 1 wherein said ALD cycles comprise introduction of water or ozone following introduction of a HfO₂ precursor.

12. A method for producing a highly sensitive, highly selective sensing chip, said method comprising:
performing a plurality of atomic layer deposition (ALD) cycles to form Al₂O₃ on a chip substrate to achieve Al₂O₃ thickness between ˜10 and 50 nm;
performing a plurality of atomic layer deposition (ALD) cycles to build on the Al₂O₃, a HfO₂ dielectric layer between about 20 and 80 nm;
annealing said chip:
applying a single wall carbon nanotube composition to form a carbon composition coating on the HfO₂ dielectric; and
applying a sensitivity functionalizing compound to the carbon composition coating.

13. The method of claim 12 wherein said layer of aluminum oxide is about 3±0.5 nm over its surface.

14. The method of claim 12 wherein said HfO₂ dielectric thickness is between about 10 nm and 70 nm.

15. The method of claim 12 wherein said HfO₂ dielectric thickness is between about 20 nm and 50 nm.

16. The method of claim 12 wherein said HfO₂ dielectric thickness is about 40 nm.

17. The method of claim 12 wherein said sensitivity functionalizing compound comprises DNA.

18. The method of claim 12 wherein said atomic layer deposition (ALD) cycles are performed in a chamber with a temperature in a range about 200±50° C.

19. The method of claim 19 wherein said ALD cycles comprise introduction of water or ozone following introduction of a HfO₂ precursor.

20. A method producing a highly sensitive, highly selective sensing chip, said method comprising performing a plurality of atomic layer deposition (ALD) cycles to coat a chip base said plurality of ALD cycles comprising:
a) a first deposition cycle of precursor A reaction to saturation and flush and precursor B reaction and flush performed in a chamber environment at about 150° C.;
b) a second deposition cycle of precursor A reaction to saturation and flush and precursor B reaction and flush performed in a chamber environment at temperature about 200° C. or higher;
c) repetition of a) and b) a number of time to bring the thin film to a desired thickness; before
d) a final deposition cycle of precursor A reaction to saturation and flush and precursor B reaction and flush performed in a chamber environment at about 150° C.
